(19) United States
(12) Patent Application Publication
(10) Pub. No.: US 2026/0120807 A1
(43) Pub. Date: Apr. 30, 2026

(54) METHOD FOR CONSTRUCTING DISEASE PREDICTION MODEL

(71) Applicants: National Central University, Taoyuan City (TW); Landseed International Hospital, Taoyuan City (TW); Phalanx Biotech Group, Inc., JUBEI CITY (TW)

(72) Inventors: Li-Jen SU, New Taipei City (TW); Jing-Hong XIAO, Taoyuan City (TW); Li-Ching WU, Taipei City (TW); Hsiao-Yen KANG, Taoyuan City (TW); Tien HSU, Taichung City (TW); Chin-Pyng WU, Taipei City (TW)

(21) Appl. No.: 19/001,091

(22) Filed: Dec. 24, 2024

(30) Foreign Application Priority Data
Oct. 29, 2024 (TW) 113141322

(51) Int. Cl. G16B 40/00; G16B 20/20; G16H 50/70

(52) U.S. Cl. CPC G16B 40/00; G16B 20/20; G16H 50/70

(57) ABSTRACT

A method for constructing a disease prediction model is provided. First, a genome-wide association study (GWAS) is conducted on patients with the target disease to identify relevant SNP loci. Next, two SNP loci are randomly selected as a first SNP combination, and a first machine learning model is trained for disease prediction, with its accuracy verified. Subsequently, the remaining SNP loci are sequentially added to the first combination to generate multiple second SNP combinations, and the corresponding disease prediction models are trained and validated. Among these second combinations, the one with the highest prediction accuracy is selected as the third combination. This process is repeated until all SNP loci are included, ultimately determining the optimal SNP target combination for the final training and prediction of the disease prediction model.

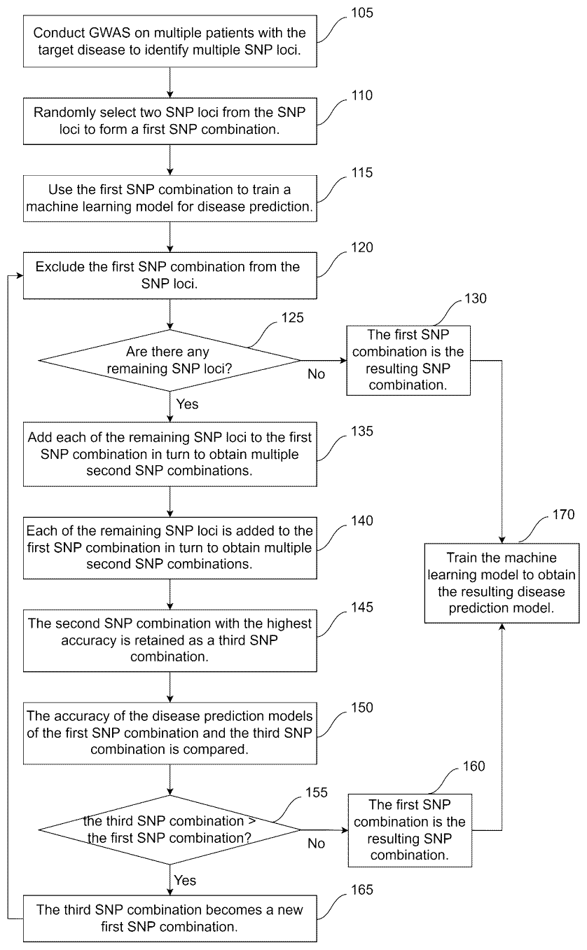

### CROSS-REFERENCE TO RELATED APPLICATION

This application claims the priority benefit of Taiwan application serial no. 113141322, filed on Oct. 29, 2024, the full disclosure of which is incorporated herein by reference.

### BACKGROUND

### Technical Field

The present invention relates to a method for constructing a disease prediction model, and more particularly to a method for constructing a disease prediction model using combinations of single nucleotide polymorphism (SNP) loci.

### Description of Related Art

Single Nucleotide Polymorphism (SNP) refers to a common sequence variation in a single region of deoxyribonucleic acid (DNA), resulting from the substitution of a single nucleotide, which contributes to genetic diversity. SNPs are the most common form of human genetic variation, accounting for more than 90% of all known genetic diversity. On average, there is one SNP per 500 to 1,000 base pairs in the human genome, with an estimated 3 million SNP variations in total. These SNP variations are stable and widely distributed across the genome, with a variation frequency typically greater than 1%. SNPs also exhibit differences among different populations, leading to genetic diversity between patients. This SNP variation affects susceptibility to various diseases. As a result, the relationship between SNPs and diseases has garnered increasing attention internationally in recent years.

However, the current approaches for applying SNPs to disease risk prediction largely focus on the relationship between a single SNP and a specific disease. Moreover, these studies are often not applicable to the majority of the Chinese population. Since many complex diseases, such as type 2 diabetes (T2D) and obesity, are influenced by multiple SNPs, research that only considers the relationship between a single SNP and a disease faces significant challenges when applied to disease risk prediction and personalized medicine.

Genome-wide association study (GWAS) is a method used to search for sequence variations (such as the aforementioned SNPs) across the human genome and perform imputation on missing sequence variation data. GWAS is particularly useful for analyzing complex diseases that rely on a combination of genetic and environmental factors. In these studies, SNPs are commonly used to locate and identify genomic regions that may contribute to common complex diseases, thus revealing gene loci associated with various diseases and improving genetic counseling for patient disease risk assessment. Therefore, GWAS provides an essential tool for studying complex diseases to aid the understanding of the association between genes and specific phenotypes. However, since most SNPs are located in non-coding regions, it is difficult to understand their functional roles. Although GWAS is effective in identifying multiple SNPs associated with various diseases, it can only analyze the relationship between a single gene and a single disease at a time, making it unsuitable for studying the synergistic effects of multiple genes and their relationship to a single disease.

Polygenic Risk Score (PRS) aims to quantify the cumulative effect of multiple genes or SNP loci by condensing the genetic variation information from several genomes into an estimate of a patient's genetic predisposition to a particular phenotype or trait. In simple terms, it is the weighted sum of the number of variant alleles (0, 1, or 2) carried by each patient, where the weights are the effect size estimates from GWAS data of the relationship between the variant alleles and the phenotype, assuming an additive genetic model. Generally, while disease prediction models built using PRS are easy to interpret, PRS often includes thousands of SNPs. Therefore, for the specific disease and population being studied, the SNPs identified by PRS may not be sufficiently accurate and thus may not be useful. Additionally, since the data in GWAS databases primarily come from European populations, the accuracy for non-European populations is even more questionable.

In summary, SNPs play a key role in genetics and personalized medicine. While GWAS and PRS have made significant progress as important tools for studying and predicting disease risk, there are still challenges in their application. Further improvements and refinements are needed to enhance their applicability and accuracy across different populations.

### SUMMARY

In one aspect, the present invention is directed to method for constructing a disease prediction model. The method comprises:
(1) conducting a genome-wide association study (GWAS) on a plurality of sample patients with a target disease to identify a plurality of single nucleotide polymorphism (SNP) loci associated with the target disease;
(2) randomly selecting two SNP loci from the identified SNP loci to form a first SNP combination;
(3) training a first machine learning model using the first SNP combination for disease prediction to generate a first disease prediction model, and then testing the prediction accuracy of the first disease prediction model for the target disease;
(4) excluding the first SNP combination from the identified SNP loci, and sequentially adding each remaining SNP locus to the first SNP combination to form a plurality of second SNP combinations;
(5) training the first machine learning model using the second SNP combinations for disease prediction to generate a plurality of second disease prediction models, and testing the prediction accuracy of each second disease prediction model for the target disease;
(6) identifying the second disease prediction model with the highest accuracy as a third disease prediction model, and designating the corresponding second SNP combination as a third SNP combination;
(7) comparing the accuracy of the first disease prediction model with the third disease prediction model;
(8) if the first disease prediction model has higher accuracy, the first SNP combination becomes a resulting SNP combination;
(9) if the third disease prediction model has higher accuracy, the third SNP combination is used as the new first SNP combination, and step (4) is repeated;
(10) when no SNP loci remain to be excluded in step (4), the new first SNP combination from step (9) becomes the resulting SNP combination;
(11) if there are still remaining SNP loci to exclude in step (4), steps (5) to (10) are repeated until the resulting SNP combination is found in either step (8) or step (10); and
(12) training the first machine learning model using the resulting SNP combination to generate a resulting disease prediction model for predicting the target disease.
According to an embodiment of this invention, the method further comprises the following steps.
(13) performing step (2) to generate a new first SNP combination;
(14) performing steps (3) to (11) to generate a new resulting SNP combination;
(15) performing step (12) to generate a new resulting disease prediction model from the first machine learning model; and
(16) comparing the accuracy of the resulting disease prediction model from step (12) with the new resulting disease prediction model from step (15), and retaining the higher accuracy model as a better SNP combination and a better disease prediction model for the first machine learning model.

According to an embodiment of this invention, the steps (13) to (16) are repeated multiple times to find an ultimate SNP combination and an ultimate disease prediction model with the highest accuracy for the first machine learning model.

According to an embodiment of this invention, the method further comprises:
(17) replacing the first machine learning model with a second machine learning model;
(18) performing steps (3) to (11) to generate the resulting SNP combination for the second machine learning model; and
(19) Performing step (12) to generate a resulting disease prediction model for the second machine learning model.

According to an embodiment of this invention, the method comprises:
(20) performing step (2) to generate a new first SNP combination;
(21) performing steps (3) to (11) to generate a new resulting SNP combination;
(22) performing step (12) to generate a new resulting disease prediction model for the second machine learning model; and
(23) comparing the accuracy of the resulting disease prediction model from step (12) with the new resulting disease prediction model from step (22), and retaining the higher accuracy model as a better SNP combination and a better disease prediction model for the second machine learning model.

According to an embodiment of this invention, the steps (15) to (17) are repeated multiple times to find the ultimate SNP combination and ultimate disease prediction model with the highest accuracy for the second machine learning model.

According to an embodiment of this invention, the first and second machine learning models comprise naive bayes (NB), library for support vector machine (libSVM), stochastic gradient descent support vector machine (SGDSVM), sequential minimal optimization logistic (SMO), k-nearest neighbors (K-NN), locally weighted learning (LWL), repeated incremental pruning to produce error reduction (PIPPER), one-rule classifier (ORC), pruning rule-based classification tree (PART), zero-rule classifier (ZRC), C4.5 decision trees (C4.5), logistic model tree (LMT), random tree (RT), random forest (RF), or any combination thereof.

According to an embodiment of this invention, a significance threshold for identifying SNP loci in the genome-wide association study in step (1) is a P-value<0.05.

According to an embodiment of this invention, the target disease includes a combination of multiple diseases.

According to an embodiment of this invention, the diseases comprise type 2 diabetes, hypertension, and ocular diseases.

As described above, the present invention provides a method for constructing a disease prediction model that can improve prediction accuracy and applicability. Through multiple iterations and comparisons of different SNP combinations, combined with various machine learning algorithms, the optimal prediction model is identified. This method is applicable to a variety of diseases, characterized by flexibility and automated optimization, effectively enhancing the predictive capability and adaptability of disease prediction models.

The foregoing presents a simplified summary of the disclosure in order to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

To make the aforementioned and other aspects, features, advantages, and embodiments of the present invention more comprehensible, the descriptions of the accompanying drawings are as follows.

The solo FIGURE is a core flowchart of the method for constructing a disease prediction model according to one embodiment of the present invention.

### DETAILED DESCRIPTION

As described above, a method for constructing a disease prediction model is provided. This method enhances the applicability and accuracy of a disease prediction model. In the following description, an exemplary construction method of the aforementioned disease prediction model will be introduced.

To provide a more comprehensive description of the implementation of the present invention, the following will offer explanatory descriptions regarding different aspects and specific embodiments. These are not limited to any one form of implementation or application but encompass the features and methodological steps of multiple specific embodiments. Different embodiments can achieve the same or similar functions and steps to demonstrate the flexibility of the present invention.

### Method for Constructing Disease Prediction Model

The FIGURE is a core flowchart of the method for constructing a disease prediction model according to one embodiment of the present invention.

In step 105, a genome-wide association study (GWAS) is conducted on a plurality of sample patients with a target disease to identify multiple single nucleotide polymorphism loci (hereinafter referred to as SNP loci) associated with the target disease. According to some embodiments of the present invention, the threshold for selecting these SNP loci using the GWAS is based on the significance level of the association between the SNP loci and the target disease, determined by a P-value<0.05. For example, the P-value could be <0.04, <0.03, <0.02, or <0.01, and the P-value can be adjusted according to the actual circumstances and requirements.

According to other embodiments of the present invention, the target disease may be a single disease or a combination of multiple related diseases (e.g., type 2 diabetes, hypertension, and eye diseases). When the target disease is a single disease, the control group for the genome-wide association study (GWAS) of sample patients with the target disease is the genomic data from a healthy population. When the target disease is a combination of multiple related diseases, the control group includes the genomic data from sample patients who have at least one of the related diseases.

Since the multiple SNP loci obtained from the genome-wide association study (GWAS) are independently selected and often include many SNP loci that may not be relevant, the next step is to apply the best path search (BPS) algorithm to filter these SNP loci. This BPS algorithm helps find the final SNP combination, containing multiple SNP loci, that is suitable for each respective machine learning model. The final SNP combination is then used to train the machine learning models to obtain the final disease prediction model, thereby improving the prediction accuracy of different machine learning models for estimating the likelihood of an individual developing the target disease in the future.

In step 110, two SNP loci are randomly selected from the multiple SNP loci obtained in step 105 to form a first SNP combination. The first SNP combination serves as the starting point for identifying an SNP combination suitable for use in the machine learning model to predict the target disease.

In step 115, the first SNP combination obtained in step 110 is used to train a machine learning model for disease prediction, resulting in a first disease prediction model. The prediction accuracy of this first disease prediction model, using the first SNP combination, is then tested for the target disease.

According to some embodiments of the present invention, the sample patients can be divided into several parts. One part is used as the training dataset for training the machine learning model, another part is used as the validation dataset to prevent overfitting of the machine learning model, and another part is used as the testing dataset to evaluate the prediction accuracy of the disease prediction model. If the likelihood of overfitting in the machine learning model's training results is low, the validation dataset can be omitted.

According to some embodiments of the present invention, the machine learning model can be, for example, naive Bayes (NB), library for support vector machine (libSVM), stochastic gradient descent support vector machine (SGDSVM), sequential minimal optimization logistic (SMO), k-nearest neighbors (K-NN), locally weighted learning (LWL), repeated incremental pruning to produce error reduction (PIPPER), one-rule classifier (ORC), pruning rule-based classification tree (PART), zero-rule classifier (ZRC), C4.5 decision trees (C4.5), logistic model tree (LMT), random tree (RT), random forest (RF), or any combination thereof.

According to other embodiments of the present invention, the validation method for the first disease prediction model includes cross-validation, such as k-fold cross-validation or leave-one-out cross-validation (LOOCV). After performing cross-validation on the first disease prediction model, the prediction accuracy of the model regarding whether a non-specific individual may develop the target disease can be improved.

In step 120, the SNP loci included in the first SNP combination are excluded from the identified SNP loci obtained in the step 105.

In step 125, it is determined whether there are any remaining SNP loci after executing step 120. If no SNP loci remain, step 130 is executed, and the first SNP combination is designated as the resulting SNP combination. If there are remaining SNP loci, step 135 is executed, where each of the remaining SNP loci is sequentially added to the first SNP combination to form multiple second SNP combinations.

In step 140, each of the second SNP combinations is used in turn to train the machine learning model for predicting the target disease, resulting in multiple corresponding second disease prediction models. The prediction accuracy of each of these second disease prediction models, using the corresponding second SNP combinations, is then tested for the target disease.

In step 145, the second disease prediction model with the highest accuracy is designated as a third disease prediction model, and the second SNP combination used in the third disease prediction model is designated as a third SNP combination.

In step 150, the accuracy of the first disease prediction model is compared with the accuracy of the third disease prediction model.

In step 155, if the comparison in step 150 shows that the accuracy of the first disease prediction model is higher, step 160 is executed, and the first SNP combination is designated as the resulting SNP combination. If the comparison shows that the third disease prediction model has higher accuracy, step 165 is executed, and the third SNP combination becomes a new first SNP combination. Next, step 120 is performed, and steps 125-165 are then repeated.

After designating the first SNP combination as the resulting SNP combination in step 130 or step 160, step 170 is executed. In step 170, the machine learning model is trained using the resulting SNP combination to obtain a resulting disease prediction model, thereby completing the first round of constructing the disease prediction model for predicting the target disease.

According to some embodiments of the present invention, if it is desired to determine whether the resulting SNP combination obtained in step 130 or step 160, and the resulting disease prediction model obtained in step 170, can be further improved, step 110 can be repeated to obtain a different new first SNP combination as a new starting point for training the machine learning model. Steps 115 to 170 are then repeated to complete the second round of constructing the disease prediction model. The accuracy of the resulting disease prediction models from the first and second rounds is then compared, and the model with the better prediction accuracy is designated as the optimal disease prediction model, with its corresponding SNP combination designated as the optimal SNP combination.

According to other embodiments of the present invention, step 110 can be repeated once more to obtain another new and different first SNP combination as a new starting point for training the machine learning model. Steps 115 to 170 are then repeated to complete a third round of constructing the disease prediction model. The accuracy of the optimal disease prediction model obtained from the second round is compared with that of the resulting disease prediction model from the third round. The model with the better prediction accuracy, whether the optimal or resulting disease prediction model, is designated as the new optimal disease prediction model. The SNP combination used by this new optimal disease prediction model becomes the new optimal SNP combination.

This process is repeated until either the prediction accuracy of the new resulting disease prediction model from the new round using the new resulting SNP combination is the same as the prediction accuracy of the optimal disease prediction model from the previous round using the optimal SNP combination, or until the executor is satisfied with the results, thus the construction of the disease prediction model for the machine learning model is completed. At this point, the disease prediction model with the highest accuracy, as determined by the machine learning model, is designated as the final disease prediction model, and the SNP combination used by this final disease prediction model is designated as the final SNP combination.

According to other embodiments, the machine learning model used in the entire process described above (referred to as the first machine learning model) can be replaced by a new machine learning model (referred to as a second machine learning model). The final disease prediction model and the final SNP combination used by the second machine learning model can then be obtained. According to other embodiments, the final SNP combination used by the second machine learning model's final disease prediction model may be the same as or different from the final SNP combination used by the first machine learning model's final disease prediction model, due to the different computational characteristics of each machine learning model.

### Method of Using the Disease Prediction Model

Since the method for constructing the disease prediction model can find the final disease prediction model and the corresponding final SNP combination respectively for multiple machine learning models. When whether a target individual will develop the target disease is predicted, the disease prediction models respectively constructed by multiple machine learning models can first be separately used to predict the individual. Each disease prediction model will provide its own prediction result.

Then, the prediction results are aggregated, and the final result for the target individual is determined using a voting-like method. If most prediction results indicate that the individual is likely to develop the target disease, it is concluded that the individual will develop the disease. Conversely, if most prediction results indicate that the individual is unlikely to develop the target disease, it is concluded that the individual will not develop the disease.

### Construction of Disease Prediction Models for Type 2 Diabetes, Hypertension, and Eye Diseases

### Study Subjects and Data Preprocessing

In this embodiment, type 2 diabetes was preliminarily selected as the target disease.

The training data for the machine learning model was sourced from approximately 520,000 outpatients at Landseed International Hospital in Taoyuan City, Taiwan, between 2007 and 2015. After excluding patients with incorrect gender information, children, and pregnant women, all patients diagnosed with type 2 diabetes were selected. Then, these type 2 diabetes patients were further examined to determine if they had at least two records of the same other disease code (ICD9) in outpatient visits within one year, identifying them as confirmed patients of other diseases. These patients, who were diagnosed with both type 2 diabetes and other diseases, were subjected to a genome-wide association study (GWAS) to identify diseases highly correlated with type 2 diabetes. The results of the GWAS are shown in Table 1.

TABLE 1
Results of the genome-wide association study (GWAS) for patients
diagnosed with both type 2 diabetes and other diseases, showing
diseases highly correlated with type 2 diabetes.

| | ICD9 | Number of | Odds | |
|---|---|---|---|---|
| Disease | Code | Patients | Ratio | P-value |
| Retinal disease | 362 | 4,066 | 11.78 | <0.05 |
| Cataracts | 366 | 3,663 | 7.94 | <0.05 |
| Essential hypertension | 401 | 14,474 | 5.76 | <0.05 |
| Chronic ischemic heart disease | 414 | 3,718 | 5.99 | <0.05 |
| Heart failure | 428 | 3,740 | 5.71 | <0.05 |
| Cerebral artery occlusion | 434 | 2,615 | 7.09 | <0.05 |
| Conjunctival diseases | 372 | 2,255 | 11.68 | <0.05 |
| Urethral and urinary tract diseases | 599 | 1,063 | 2.27 | <0.05 |
| Sequelae of cerebrovascular disease | 438 | 1,340 | 7.39 | <0.05 |
| Chronic renal failure | 585 | 1,486 | 6.22 | <0.05 |
| Cardiac arrhythmia | 427 | 1,009 | 8.83 | <0.05 |

From the results in Table 1, it is evident that hypertension has the highest correlation with type 2 diabetes. Patients with both type 2 diabetes and hypertension represent the most common disease combination recorded at Landseed International Hospital, accounting for approximately 23%. The combination of type 2 diabetes and eye diseases (retinal disease and cataracts) showed the highest odds ratio, indicating that the risk of eye diseases significantly increases after the onset of type 2 diabetes, with a multiplicative effect exceeding eightfold. To determine the interrelationship between different diseases, a chi-square test was used to verify the statistical significance of the associations between diseases, with all P-values being <0.05. Therefore, in this embodiment, the target disease was selected as the combination of type 2 diabetes, hypertension, and eye diseases. Next, from the confirmed patients at Landseed International Hospital, 440 samples of patients diagnosed with at least type 2 diabetes, hypertension, or eye diseases were selected.

To reduce genotyping analysis errors caused by population differences, the largest group, the Hakka population, was selected as the focus for the subsequent GWAS, comprising a total of 242 Hakka samples. From these 242 Hakka samples, 84 DNA samples were randomly selected from patients who had at least two of the target diseases for the subsequent GWAS. Additionally, 16 DNA samples from healthy individuals were selected as the control group for the GWAS. Therefore, a total of 100 DNA samples (84 from patients and 16 from healthy individuals) were collected. After excluding low-quality DNA samples, such as those showing signs of degradation, a total of 96 DNA samples remained. These were analyzed using the Axiom Genome-Wide TWB 2.0 Array Plate, which contains approximately 686,463 SNPs, to conduct the GWAS.

The 96 DNA samples mentioned above were compared with 8,287 Hakka samples retrieved from version 2.0 of the Taiwan Biobank (TW Biobank, TWB). Following quality control procedures, the PLINK (v1.9) toolkit was used to perform quality control (QC) on the DNA samples and the SNP markers obtained through GWAS. This process excluded any potential errors or poor data in the DNA samples and SNP markers to ensure the quality and accuracy of the raw data. The QC criteria for the GWAS and the exclusion standards for DNA samples used in this study are listed in Table 2. The QC processing steps are described as follows:
(1) The data is filtered based on the missing data rate.
(2) Gender is confirmed.
(3) Minor alleles are filtered.
(4) The Hardy-Weinberg equilibrium test is performed.
(5) Heterogeneity is filtered.
(6) Individuals with familial relationships are excluded.

TABLE 2
GWAS quality control (QC) items and
exclusion criteria for DNA samples.

| QC Item | Standard Value |
|---|---|
| Missing detection rate | >2% |
| Males with an impurity rate | <0.2 |
| Females with an impurity rate | >0.8 |
| Minor allele frequency (MAF) | <5% |
| Hardy-Weinberg equilibrium | p < 1.0 × 10⁻⁶ |
| Overall genome impurity rate | 99.7% confidence interval* |
| Identity by descent (IBD) per individual | >0.1875** |

*Patients with a homozygous rate that exceeds this confidence interval are considered abnormal and excluded.
**0.1875 is the expected average IBD value for second- and third-degree relatives. Individuals with an IBD >0.1875 are considered to have cryptic relatedness, and the individual with the lowest missing detection rate is retained.

After quality control filtering, 96 Hakka DNA samples from Landseed International Hospital and 267,679 SNP variants from 8,287 Hakka participants in the TWB 2.0 of the Taiwan Biobank were retained.

The testing dataset for the machine learning models was sourced from the Taiwan Biobank TWB 2.0. As of September 2022, the Taiwan Biobank had genotyped 103,252 participants using the custom TWB 2.0 chip.

In the following embodiments and comparative examples, 14 different machine learning models will be used. The English names of these models are listed in Table 3 below.

TABLE 3
Names of 14 different machine learning models.

| | English |
|---|---|
| English Name | Abbreviation |
| Naive Bayes | NB |
| Library for Support Vector Machines | libSVM |
| Stochastic Gradient Descent Support Vector Machine | SGD |
| Sequential Minimal Optimization Logistic | SMO |
| K-Nearest Neighbors | K-NN |
| Locally Weighted Learning | LWL |
| Repeated incremental pruning to produce error reduction | PIPPER |
| One-Rule Classifier | ORC |
| Pruning rule-based classification tree | PART |
| Zero-Rule Classifier | ZRC |
| C4.5 Decision Trees | C4.5 |
| Logistic Model Tree | LMT |
| Random Tree | RT |
| Random Forest | RF |

### Comparative Example 1: Training Data for Machine Learning Models from GWAS Data (P-value<0.0001)

In the results of the GWAS obtained in Comparative Example 1, a significance threshold of P-value<0.0001 was used to filter the SNP loci associated with the three diseases: type 2 diabetes, hypertension, and eye diseases. The 14 machine learning models listed in Table 3 were then applied to learn and assess the relationship between these SNP loci and the likelihood of developing the target diseases. As a result, 52 significantly different SNP loci were identified. Among these 52 SNP loci, 10 were related to diabetes, 27 were related to eye diseases, and 23 were related to hypertension. Additionally, 5 loci were shared between diabetes and hypertension, and 3 loci were shared between eye diseases and hypertension.

To reduce the possibility of overfitting, the leave-one-out cross-validation (LOOCV) algorithm was adopted to enhance the predictive ability of the disease prediction models generated from training the machine learning models. Ultimately, the accuracy of the disease prediction models of the Hakka population from Pingzhen, Taoyuan, was tested using a dataset from the Taiwan Biobank TWB 2.0, which comes from the same region. To improve the prediction accuracy of the machine learning models, SNP loci with high influence on the prediction of the target diseases were identified and retained, while redundant SNP loci with low influence were excluded. The results are shown in Table 4.

TABLE 4
Prediction accuracy of 14 disease prediction models
constructed using SNP loci from GWAS with P-value <
0.0001. These accuracies were obtained by testing each
model with data from the Taiwan Biobank TWB 2.0. The GWAS
selection threshold was set at P-value < 10⁻⁴

| Machine | Accuracy (%) | | | | | |
|---|---|---|---|---|---|---|
| Learning | Diabetes | | Eye Diseases | | Hypertension | |
| Model | GWAS | TWB | GWAS | TWB | GWAS | TWB |
| C4.5 | 69.79 | 57.69 | 43.75 | 75.00 | 42.71 | 48.48 |
| K-NN | 82.29 | 57.69 | 58.33 | 64.29 | 62.5 | 48.48 |
| libSVM | 91.67 | 53.85 | 64.58 | 57.14 | 56.25 | 48.48 |
| LMT | 87.50 | 61.54 | 62.50 | 57.14 | 50.00 | 39.39 |
| LWL | 50.00 | 46.15 | 60.42 | 60.71 | 47.92 | 51.52 |
| NB | 88.54 | 61.54 | 56.25 | 60.71 | 46.88 | 36.36 |
| ORC | 51.04 | 46.15 | 50.00 | 60.71 | 62.50 | 45.45 |
| PART | 68.75 | 42.31 | 48.96 | 60.71 | 43.75 | 45.45 |
| RF | 84.38 | 69.23 | 59.38 | 67.86 | 51.04 | 45.45 |
| RT | 77.08 | 65.38 | 55.21 | 46.43 | 54.17 | 36.36 |
| PIPPER | 78.13 | 57.69 | 59.38 | 60.71 | 48.96 | 45.45 |
| SGD | 89.58 | 50.00 | 56.25 | 60.71 | 41.67 | 45.45 |
| SMO | 79.17 | 57.69 | 60.42 | 64.29 | 42.71 | 42.42 |
| ZRC | 52.08 | 61.54 | 64.58 | 57.14 | 56.25 | 48.48 |

From the results in Table 4, it can be observed that for the type 2 diabetes prediction model, the library for support vector machine (libSVM) achieved an accuracy of 91.67% during cross-validation, but this accuracy dropped to 53.85% when tested with the Taiwan Biobank TWB 2.0 dataset. This decrease suggests that while the type 2 diabetes prediction model fits well with the DNA samples from Landseed International Hospital, its prediction accuracy is limited when applied to the Taiwan Biobank TWB 2.0 dataset. On the other hand, although the random forest (RF) model did not perform the best in cross-validation (84.38%), it showed the highest accuracy (69.23%) when tested with the Taiwan Biobank TWB 2.0 dataset.

For the eye disease prediction model, both the library for support vector machine (libSVM) and the zero-rule classifier (ZRC) achieved the best results in cross-validation, with an accuracy of 64.58%. However, when testing the disease prediction models using the Taiwan Biobank TWB 2.0 dataset, the C4.5 decision tree (C4.5) displayed the highest accuracy (75%), despite only achieving 43.75% accuracy during cross-validation with the hospital DNA samples. This result indicates that the predictive capability of the C4.5 decision tree model for eye diseases is limited.

For the hypertension prediction model, both the k-nearest neighbors algorithm (K-NN) and the one-rule classifier (ORC) achieved the best results in cross-validation, with an accuracy of 62.5%. However, when testing the disease prediction models using the Taiwan Biobank TWB 2.0 dataset, the locally weighted learning (LWL) model showed the highest accuracy (51.52%).

From the above results, it is evident that even when using GWAS to identify SNP loci significantly associated with the target diseases, the test results of the disease prediction models obtained from various machine learning models were not very satisfactory.

### Comparative Example 2: Training Data for Machine Learning Models from GWAS Data

To further improve the disease prediction results from Comparative Example 1, it would be necessary to use a larger number of DNA samples and analyze more SNP loci. Therefore, in Comparative Example 2, the number of SNP loci used for disease prediction in the machine learning models was increased. All SNP loci identified by GWAS were included, and after performing quality control on these SNP loci, a total of 267,679 SNP loci were obtained to serve as the feature pool for model construction and cross-validation.

In the results of Comparative Example 2, the training outcomes of the machine learning models were as follows: the highest accuracy of the type 2 diabetes prediction model (One-Rule Classifier) reached 76.04%, the highest accuracy of the eye disease prediction model (One-Rule Classifier) reached 77.08%, and the highest accuracy of the hypertension prediction model (Locally Weighted Learning) reached 75.00%. However, after validating the disease prediction models obtained from the various machine learning models using the Taiwan Biobank TWB 2.0 dataset, the results were less than satisfactory. This may be due to the excessive number of SNP loci used in Comparative Example 2, which likely caused issues with model convergence, preventing effective improvement in the prediction accuracy of the disease models.

Furthermore, compared to the training results in Comparative Example 1, the training results in Comparative Example 2 showed that the best prediction accuracy for the type 2 diabetes model decreased from 91.67% to 76.04%, while the best prediction accuracy for the eye disease model increased from 64.58% to 77.08%, and the best prediction accuracy for the hypertension model increased from 62.5% to 75%.

### Comparative Example 3: Training Data for Machine Learning Models from GWAS Data (P-value<0.01)

In Comparative Example 3, to balance retaining disease-associated SNPs and avoiding excessive noise, a P-value<0.01 was used as the threshold. From the SNP loci identified by GWAS, 5,973 SNP loci associated with type 2 diabetes, eye diseases, and hypertension were selected to train the various machine learning models.

Compared to the disease prediction models constructed using the entire SNP pool (267,679 SNP loci) in Comparative Example 2, the disease prediction models in Comparative Example 3 showed higher accuracy, although some models experienced slight decreases in accuracy due to algorithmic rules. However, the test results using the Taiwan Biobank TWB 2.0 dataset were still unsatisfactory. It is possible that the number of SNP loci used was still too large, hindering the convergence of the disease prediction models, thereby limiting their ability to effectively improve prediction accuracy.

### Example: Using the Best Path Search Algorithm to Select the Smallest and Most Effective SNP Loci Combination

In this example, the same SNP selection criteria as in Comparative Example 3 were used, where the GWAS results with a P-value<0.01 were set as the threshold. A total of 5,973 SNP loci associated with type 2 diabetes, eye diseases, and hypertension were selected.

Next, the best path search (BPS) algorithm was used to select the most effective and smallest SNP loci combinations (hereafter referred to as SNP combinations) for each machine learning model to predict the three aforementioned diseases. For details on the best path search algorithm, please refer to the relevant description of steps 110-170 in the FIGURE, which will not be repeated here.

Then, cross-validation was performed on the previously trained disease prediction models to prevent overfitting of the machine learning models to the SNP combinations. The cross-validation algorithm used here is the k-fold cross-validation algorithm.

In human SNP datasets, confounding factors such as population stratification and cryptic relatedness may introduce false associations. To reduce this effect, cross-validation techniques were employed. Multiple machine learning models are trained on different subsets of the same structured training data and evaluating them on independent validation data. Additionally, to assess the importance of SNP features, the importance of each feature was evaluated across all models constructed during cross-validation to prevent false associations. By adopting this model construction approach, prediction accuracy is improved while ensuring that relevant biomarkers are selected, with the goal of discovering methods to enhance the accuracy of disease prediction models, even with small sample datasets.

Finally, the disease prediction models generated by each machine learning model after cross-validation were tested using the Taiwan Biobank TWB 2.0 dataset. The test results are shown in Table 5. As seen from Table 5, among the various disease prediction models tested, the random forest model using the best path search (BPS) consistently achieved over 88% cross-validation accuracy across all three diseases and exceeded 85% accuracy when tested with the Taiwan Biobank TWB 2.0 dataset. Through training with the random forest model, the final SNP combination related to type 2 diabetes, eye diseases, and hypertension was selected. The final SNP combination of the random forest disease prediction model includes 39 SNP loci: 14 SNP loci for type 2 diabetes, 10 SNP loci for eye diseases, and 15 SNP loci for hypertension. The details of these SNP loci in the final SNP combination are listed in Table 6.

TABLE 5
This table presents the results of using machine learning to select SNP loci and build models under
different conditions, along with the cross-validation results and the testing results on the Taiwan
Biobank dataset. The total number of SNP loci is 267,679. The GWAS selection results include 2,848
SNP loci for type 2 diabetes, 2,878 SNP loci for cataracts, and 2,883 SNP loci for hypertension.

| | SNP selection criteria | | | | | | | | | | | |
|---|---|---|---|---|---|---|---|---|---|---|---|---|
| | Diabetes (%) | | | | Eye Diseases (%) | | | | Hypertension (%) | | | |
| Model | All | | | | All | | | | All | | | |
| Names | Loci | GWAS | BPS | TWB | Loci | GWAS | BPS | TWB | Loci | GWAS | BPS | TWB |
| C4.5 | 45.83 | 43.75 | 95.83 | 65.38 | 50 | 63.54 | 92.71 | 46.43 | 57.29 | 48.96 | 93.75 | 57.58 |
| | | | (8 SNPs) | | | | (12 SNPs) | | | | (7 SNPs) | |
| K-NN | 46.88 | 100 | 97.92 | 61.54 | 54.17 | 100 | 97.92 | 35.71 | 46.88 | 100 | 100 | 60.61 |
| | | | (7 SNPs) | | | | (9 SNPs) | | | | (9 SNPs) | |
| libSVM | 52.08 | 100 | 95.83 | 65.38 | 64.58 | 100 | 100 | 53.57 | 56.25 | 100 | 96.88 | 66.67 |
| | | | (10 SNPs) | | | | (12 SNPs) | | | | (9 SNPs) | |
| LMT | 60.42 | 56.25 | 100 | 65.38 | 59.38 | 75 | 100 | 50 | 58.33 | 62.5 | 96.88 | 54.55 |
| | | | (10 SNPs) | | | | (11 SNPs) | | | | (7 SNPs) | |
| LWL | 69.79 | 62.5 | 89.58 | 42.31 | 57.29 | 82.29 | 87.5 | 53.57 | 75 | 60.42 | 94.79 | 39.39 |
| | | | (10 NPs) | | | | (5 SNPs) | | | | (7 SNPs) | |
| NB | 40.63 | 100 | 100 | 65.38 | 64.58 | 100 | 98.96 | 60.71 | 56.25 | 100 | 97.92 | 51.52 |
| | | | (9 SNPs) | | | | (7 SNPs) | | | | (7 SNPs) | |
| ORC | 76.04 | 23.96 | — | — | 77.08 | 63.54 | — | — | 57.29 | 71.88 | — | — |
| PART | 55.21 | 54.17 | 96.88 | 57.69 | 56.25 | 62.5 | 88.54 | 60.71 | 67.71 | 53.13 | 93.75 | 66.67 |
| | | | (9 SNPs) | | | | (6 SNPs) | | | | (9 SNPs) | |
| RF | 46.88 | 96.88 | 93.75 | 88.46 | 63.54 | 85.42 | 88.54 | 85.71 | 54.17 | 94.79 | 90.63 | 87.88 |
| | | | (14 SNPs) | | | | (10 SNPs) | | | | (15 SNPs) | |
| RT | 45.83 | 67.71 | 94.79 | 53.85 | 54.17 | 69.79 | 94.79 | 57.14 | 59.38 | 66.67 | 95.83 | 48.48 |
| | | | (6 SNPs) | | | | (7 SNPs) | | | | (7 SNPs) | |
| PIPPER | 57.29 | 47.92 | 95.8 | 50 | 57.29 | 62.5 | 95.83 | 39.29 | 53.13 | 58.33 | 93.75 | 63.64 |
| | | | (7 SNPs) | | | | (8 SNPs) | | | | (7 SNPs) | |
| SGD | 54.17 | 100 | 97.92 | 65.38 | 63.54 | 100 | 98.96 | 57.14 | 57.29 | 100 | 98.96 | 63.64 |
| | | | (8 SNPs) | | | | (7 SNPs) | | | | (8 SNPs) | |
| SMO | 43.75 | 100 | 98.96 | 53.85 | 64.58 | 100 | 96.88 | 53.57 | 56.25 | 100 | 97.92 | 54.55 |
| | | | (6 SNPs) | | | | (5 SNPs) | | | | (8 SNPs) | |
| ZRC | 52.08 | 52.08 | — | — | 64.58 | 64.58 | — | — | 56.25 | 56.25 | — | — |

TABLE 6
This table presents the SNP loci related to type 2 diabetes, eye diseases, and hypertension, selected by the random forest model
using the best path search algorithm from Table 5. The SNP loci with a P-value < 0.01 from GWAS are the same as those
selected in Comparative Example 3, while the SNP loci with a P-value < 0.0001 from GWAS are the same as those selected
in Comparative Example 1. The intersecting columns indicate SNP loci selected in both Comparative Example 1 and this embodiment.

| | | | | | | | | GWAS (P-value) | | | |
|---|---|---|---|---|---|---|---|---|---|---|---|
| Disease | | chromo- | | Physical | Allele | | Associate | | Eye | Hyper- | Inter- |
| name | ID | some | cytoband | position | Ref | Alt | Gene | Diabetes | disease | tension | section ^{#} |
| Diabetes | rs12044674 | 1 | q23.3 | 164909595 | G | T | PBX1, | * | * | * | — |
| | | | | | | | LMX1A | | | | |
| | rs12121653 | 1 | q32.1 | 202797643 | T | C | KDM5B | * | * | ** | — |
| | rs12568685 | 1 | p31.3 | 63243590 | G | A | — | * | 0.01 | * | — |
| | rs956386 | 2 | q24.3 | 163575193 | G | T | FIGN, | * | 0.04 | 0.02 | — |
| | | | | | | | KCNH7 | | | | |
| | rs4402787 | 2 | q12.2 | 105715048 | A | G | NCK2 | * | 0.02 | 0.02 | — |
| | rs116971879 | 3 | q13.13 | 109211939 | A | C | DPPA2 | ** | ** | *** | — |
| | rs4701523 | 5 | p14.1 | 26139029 | A | T | CDH9 | * | 0.01 | * | — |
| | rs879045 | 7 | p11.2 | 56281912 | A | C | NUPR2 | * | 0.03 | * | — |
| | rs17088590 | 8 | p21.3 | 22726396 | C | T | PEBP4 | * | 0.03 | * | — |
| | rs117705722 | 9 | p22.2 | 17832199 | G | C | SH3GL2, | * | 0.15 | 0.06 | — |
| | | | | | | | ADAMTSL1 | | | | |
| | rs117174344 | 10 | p11.21 | 35758665 | C | T | FZD8, | ** | * | * | — |
| | | | | | | | PCAT5 | | | | |
| | rs117705386 | 13 | q12.3 | 29059579 | G | T | MTUS2 | * | 0.04 | * | — |
| | rs9569458 | 13 | q21.1 | 56358644 | C | T | PRR20B | * | 0.03 | * | — |
| | rs73584602 | 13 | q33.1 | 103428687 | G | A | DAOA-AS1 | * | * | * | — |
| Eye | rs6676790 | 1 | q23.1 | 157191556 | C | T | ETV3, | 0.04 | * | 0.02 | — |
| disease | | | | | | | FCRL5 | | | | |
| | rs631450 | 1 | q31.2 | 191537856 | C | T | RGS18 | * | * | * | — |
| | rs12756914 | 1 | q41 | 223053611 | A | G | DISP1, | * | * | * | — |
| | | | | | | | TLR5 | | | | |
| | rs10490598 | 2 | q36.3 | 228415811 | C | T | SPHKAP, | 0.11 | * | 0.08 | — |
| | | | | | | | PID1 | | | | |
| | rs10036055 | 5 | p14.2 | 23372581 | C | A | CDH12, | * | * | ** | — |
| | | | | | | | PRDM9 | | | | |
| | rs116941872 | 7 | q35 | 148029125 | G | T | CNTNAP2 | ** | *** | ** | V |
| | rs10100105 | 8 | q24.22 | 134045230 | G | A | ZFAT | * | * | ** | — |
| | rs6491129 | 13 | q12.13 | 26484782 | C | T | WASF3, | ** | ** | ** | — |
| | | | | | | | CDK8 | | | | |
| | rs8022707 | 14 | q12 | 25457807 | C | A | STXBP6, | * | ** | * | — |
| | | | | | | | NOVA1 | | | | |
| | rs11700536 | 21 | q22.3 | 43138577 | C | T | — | 0.09 | * | 0.05 | — |
| Hyper- | rs146599921 | 1 | q25.3 | 182001927 | AT | — | ZNF648, | 0.01 | 0.02 | * | — |
| tension | | | | | | | CACNA1E | | | | |
| | rs75282567 | 2 | p12 | 75266497 | C | A | TACR1, | 0.01 | 0.02 | * | — |
| | | | | | | | EVA1A | | | | |
| | rs75539603 | 2 | q31.1 | 173119285 | G | T | ZAK | * | 0.01 | * | — |
| | rs73058503 | 3 | p22.2 | 39205794 | A | G | XIRP1 | * | ** | ** | — |
| | rs16877783 | 6 | p22.3 | 16274973 | A | C | GMPR | 0.02 | 0.07 | * | — |
| | rs7839529 | 8 | q23.3 | 113994845 | A | C | TRPS1, | * | 0.04 | * | — |
| | | | | | | | CSMD3 | | | | |
| | rs12359245 | 10 | p14 | 7781022 | C | T | KIN | 0.01 | 0.02 | * | — |
| | rs150643536 | 13 | q21.2 | 60415465 | A | G | TDRD3 | * | 0.05 | * | — |
| | rs9514209 | 13 | q31.2 | 88032696 | A | C | — | * | * | * | — |
| | rs77411406 | 13 | q33.1 | 101414500 | C | T | NALCN | * | 0.03 | * | — |
| | rs2333236 | 14 | q12 | 28271592 | G | A | FOXG1-AS1 | 0.01 | 0.02 | * | — |
| | rs74558040 | 14 | q32.2 | 97031446 | G | A | — | ** | * | ** | — |
| | rs77245215 | 16 | p12.2 | 23399047 | G | A | COG7 | 0.01 | 0.02 | * | — |
| | rs6500596 | 16 | p13.3 | 4420026 | G | T | CORO7, | * | ** | * | — |
| | | | | | | | CORO7-PAM16 | | | | |
| | rs191212406 | X | q23 | 113876824 | C | T | XACT | * | * | * | — |

* P < 0.01;
** P < 0.001;
*** P < 0.0001
^{#} Whether there is an intersection with the GWAS result (P < 0.0001).

In Table 6, it can be observed that many SNP loci in the GWAS analysis have P-values significantly greater than 0.01 (with the largest being 0.15). In traditional GWAS analysis, these SNP loci would typically not be selected. However, by using machine learning models and the best path search algorithm to construct the disease prediction model, these SNP loci were included. Furthermore, in the “Intersection” column, only one SNP locus related to “eye diseases” was selected by both Comparative Example 1 and this Example.

Finally, a comparison was made between the prediction accuracy of the 14 models constructed using the SNP combinations selected by the traditional GWAS method (P-value<0.0001, as in Comparative Example 1) and the SNP combinations selected using machine learning (i.e., using BPS). The results are shown in Table 7.

TABLE 7
Prediction accuracy of 14 machine learning models for
type 2 diabetes, eye diseases, and hypertension.

| Machine | | | | | | |
|---|---|---|---|---|---|---|
| Learning | Type 2 Diabetes | | Eye Diseases | | Hypertension | |
| Model | GWAS | BPS | GWAS | BPS | GWAS | BPS |
| NB | 61.54 | 65.38 | 56.25 | 60.71 | 36.36 | 51.52 |
| libSVM | 53.85 | 65.38 | 64.58 | 53.57 | 48.48 | 66.67 |
| SGD | 50 | 65.38 | 56.25 | 57.14 | 45.45 | 63.64 |
| SMO | 57.69 | 53.85 | 60.42 | 53.57 | 42.42 | 54.55 |
| K-NN | 57.69 | 61.54 | 58.33 | 35.71 | 48.48 | 60.61 |
| LWL | 46.15 | 42.31 | 60.42 | 53.57 | 51.52 | 39.39 |
| PIPPER | 57.69 | 50 | 59.38 | 39.29 | 45.45 | 63.64 |
| ORC | 46.15 | 23.96 | 50 | 63.54 | 45.45 | 71.88 |
| PART | 42.31 | 57.69 | 48.96 | 60.71 | 45.45 | 66.67 |
| ZRC | 61.54 | 54.17 | 64.58 | 64.58 | 48.48 | 56.25 |
| C4.5 | 57.69 | 65.38 | 43.75 | 46.43 | 48.48 | 57.58 |
| LMT | 61.54 | 65.38 | 62.5 | 50 | 39.39 | 54.55 |
| RT | 65.38 | 53.85 | 55.21 | 57.14 | 36.36 | 48.48 |
| RF | 69.23 | 88.46 | 59.38 | 85.71 | 45.45 | 87.88 |

As shown in Table 7, compared to the SNP combinations selected by traditional GWAS, most of the disease prediction models constructed using SNP combinations selected by machine learning showed improved prediction accuracy, with the random forest model exhibiting the greatest improvement.

However, for the Locally Weighted Learning (LWL) model, the SNP combination selected by traditional GWAS performed better, as the LWL algorithm is more sensitive to outliers. The SNP combination selected through GWAS effectively excluded outliers, but the highest accuracy achieved for the three diseases was only 60.71%.

As noted above, in genome-wide association studies (GWAS), the larger the sample size, the more reliable the statistical results, allowing for smaller P-values. A smaller P-value indicates a stronger statistical association between an SNP and a disease. Therefore, in studies with large sample sizes, researchers can often identify SNP loci with extremely small P-values. However, the sample size used in the embodiments of this invention is smaller than that of European and American databases, resulting in relatively larger P-values. In contrast, GWAS analyses from European and American databases often achieve P-values smaller than 5×10⁻⁸, and such SNP loci are considered statistically more significant.

To overcome the limitation of insufficient sample size, the embodiment of this invention combines the best path search algorithm with machine learning models and relaxes the P-value threshold in GWAS, selecting SNP loci with P-values less than 0.01 for analysis. The results show that the accuracy of the disease prediction models constructed using this strategy is actually higher than that of models built using only SNP loci with P-values smaller than 0.0001. This suggests that even with larger P-values, constructing disease prediction models using optimized SNP combinations can still achieve higher prediction accuracy. Moreover, while maintaining high prediction accuracy, the best path search algorithm allows for disease prediction using the smallest possible SNP combination, serving as the optimal biomarker set for identifying diseases.

As described above, the method for constructing a disease prediction model provided by this invention has the following advantages:
Improved Prediction Accuracy: By repeatedly filtering and optimizing SNP combinations and combining different machine learning models for prediction, the final model achieves higher prediction accuracy.
Enhanced Model Applicability: The constructed disease prediction model can be applied to the prediction of various diseases, such as type 2 diabetes, hypertension, and eye diseases. The model can be adjusted for different diseases, thus expanding its applicability.
Flexibility and Automation: The method allows the use of multiple machine learning algorithms and automates multiple iterations of optimization, reducing manual intervention and improving the efficiency of machine learning model training.
Comprehensive Genomic Analysis: By utilizing genome-wide association studies (GWAS) and the best path search algorithm to filter SNP loci related to the target disease, the resulting disease prediction model effectively improves prediction accuracy while relying on the minimum number of SNP loci.

Although the invention has been disclosed through the above embodiments, it is not intended to limit the invention. Any person skilled in the art can make various modifications and refinements without departing from the spirit and scope of the invention. Therefore, the scope of protection for this invention shall be defined by the appended claims.

What is claimed is:

1. A method for constructing a disease prediction model, comprising:
(1) conducting a genome-wide association study (GWAS) on a plurality of sample patients with a target disease to identify a plurality of single nucleotide polymorphism (SNP) loci associated with the target disease;
(2) randomly selecting two SNP loci from the identified SNP loci to form a first SNP combination;
(3) training a first machine learning model using the first SNP combination for disease prediction to generate a first disease prediction model, and then testing the prediction accuracy of the first disease prediction model for the target disease;
(4) excluding the first SNP combination from the identified SNP loci, and sequentially adding each remaining SNP locus to the first SNP combination to form a plurality of second SNP combinations;
(5) training the first machine learning model using the second SNP combinations for disease prediction to generate a plurality of second disease prediction models, and testing the prediction accuracy of each second disease prediction model for the target disease;
(6) identifying the second disease prediction model with the highest accuracy as a third disease prediction model, and designating the corresponding second SNP combination as a third SNP combination;
(7) comparing the accuracy of the first disease prediction model with the third disease prediction model;
(8) if the first disease prediction model has higher accuracy, the first SNP combination becomes a resulting SNP combination;
(9) if the third disease prediction model has higher accuracy, the third SNP combination is used as the new first SNP combination, and step (4) is repeated;
(10) when no SNP loci remain to be excluded in step (4), the new first SNP combination from step (9) becomes the resulting SNP combination;
(11) if there are still remaining SNP loci to exclude in step (4), steps (5) to (10) are repeated until the resulting SNP combination is found in either step (8) or step (10); and
(12) training the first machine learning model using the resulting SNP combination to generate a resulting disease prediction model for predicting the target disease.

2. The method of claim 1, further comprising:
(13) performing step (2) to generate a new first SNP combination;
(14) performing steps (3) to (11) to generate a new resulting SNP combination;
(15) performing step (12) to generate a new resulting disease prediction model from the first machine learning model; and
(16) comparing the accuracy of the resulting disease prediction model from step (12) with the new resulting disease prediction model from step (15), and retaining the higher accuracy model as a better SNP combination and a better disease prediction model for the first machine learning model.

3. The method of claim 2, wherein steps (13) to (16) are repeated multiple times to find an ultimate SNP combination and an ultimate disease prediction model with the highest accuracy for the first machine learning model.

4. The method of claim 1, further comprising:
(17) replacing the first machine learning model with a second machine learning model;
(18) performing steps (3) to (11) to generate the resulting SNP combination for the second machine learning model; and
(19) Performing step (12) to generate a resulting disease prediction model for the second machine learning model.

5. The method of claim 4, further comprising:
(20) performing step (2) to generate a new first SNP combination;
(21) performing steps (3) to (11) to generate a new resulting SNP combination;
(22) performing step (12) to generate a new resulting disease prediction model for the second machine learning model; and
(23) comparing the accuracy of the resulting disease prediction model from step (12) with the new resulting disease prediction model from step (22), and retaining the higher accuracy model as a better SNP combination and a better disease prediction model for the second machine learning model.

6. The method of claim 5, wherein steps (15) to (17) are repeated multiple times to find the ultimate SNP combination and ultimate disease prediction model with the highest accuracy for the second machine learning model.

7. The method of claim 4, wherein the second machine learning model comprises naive bayes (NB), library for support vector machine (libSVM), stochastic gradient descent support vector machine (SGDSVM), sequential minimal optimization logistic (SMO), k-nearest neighbors (K-NN), locally weighted learning (LWL), repeated incremental pruning to produce error reduction (PIPPER), one-rule classifier (ORC), pruning rule-based classification tree (PART), zero-rule classifier (ZRC), C4.5 decision trees (C4.5), logistic model tree (LMT), random tree (RT), random forest (RF), or any combination thereof.

8. The method of claim 1, wherein the first machine learning model comprises naive bayes (NB), library for support vector machine (libSVM), stochastic gradient descent support vector machine (SGDSVM), sequential minimal optimization logistic (SMO), k-nearest neighbors (K-NN), locally weighted learning (LWL), repeated incremental pruning to produce error reduction (PIPPER), one-rule classifier (ORC), pruning rule-based classification tree (PART), zero-rule classifier (ZRC), C4.5 decision trees (C4.5), logistic model tree (LMT), random tree (RT), random forest (RF), or any combination thereof.

9. The method of claim 1, wherein a significance threshold for identifying SNP loci in the genome-wide association study in step (1) is a P-value<0.05.

10. The method of claim 1, wherein the target disease includes a combination of multiple diseases.

11. The method of claim 10, wherein the diseases comprise type 2 diabetes, hypertension, and ocular diseases.
